(19) United States
(12) Patent Application Publication
(10) Pub. No.: US 2024/0201153 A1
(43) Pub. Date: Jun. 20, 2024

(54) COMPOSITE SUBSTRATE FOR OPTICAL-BASED VOC DETECTION

(71) Applicant: Analog Devices, Inc., Wilmington, MA (US)

(72) Inventors: Max Marcel Shulaker et al., Cambridge, MA (US); Samuel H Fuller, Wellesley, MA (US)

(21) Appl. No.: 18/391,332

(22) Filed: Dec. 20, 2023

Related U.S. Application Data
(60) Provisional application No. 63/476,318, filed on Dec. 20, 2022.

(51) Int. Cl. G01N 33/00; G01N 21/78

(52) U.S. Cl. CPC G01N 33/0047; G01N 21/783

(57) ABSTRACT

This document describes a composite substrate for optical detection of a target gas composition within ambient gas in an environment. A composite substrate can include a porous polymer material incorporating a second material, and the second material can include at least one chemical property to establish or adjust a hydrophobicity of the porous polymer material. The composite substrate can also include at least one functionalized region, included such as to be exposed to the ambient gas the functionalized region including at least one electrical or optical property correlative of the target gas composition.

### CLAIM OF PRIORITY

This application claims priority to U.S. Provisional Application Ser. No. 63/476,318, filed on Dec. 20, 2022, which is incorporated by reference herein in its entirety, and the benefit of priority of which is claimed herein.

### BACKGROUND

Detecting toxic and nontoxic gases is important for applications such as environmental protection, healthcare, and for potential household and industrial applications. Gases of interest can include oxygen (O2), carbon monoxide (CO), carbon dioxide (CO2), sulfur dioxide (SO2), nitrogen oxides (NO and NO2), hydrogen sulfide (H2S), methane (CH4), ethanol (C2H5OH), and propane (C3H8). A gas detector can be constructed including one or more materials that can be selected for their ability to sense one or more target gas analytes, as well as for their characteristics such as physical and chemical stability in different atmospheres or environments.

### SUMMARY

A composite substrate, such as a material used for gas chemical sensing, can include a functionalization chemical applied to the substrate such as to form a functionalized region. The functionalization chemical can be selected such as to include an optical property indicative of the target gas composition. For example, optical property can be associated with a change of the functionalized region to distinguish the target gas composition from other ambient gases. It can be desirable to utilize a coarseness or porosity of a surface of the functionalized region such as to help increase sensitivity of the gas chemical detector. For example, a porous functionalized region can increase the surface area available for chemical interaction with ambient gas and can result in increased rates of reaction with gas components indicative of a particular target gas composition. As such, the substrate can incorporate a second additive material including at least one chemical property to establish or adjust a hydrophobicity or porosity of the substrate. In an example, at least one functionalized region can be applied to the substrate including the second additive material. Here, the at least one functionalized region can be included such as to be exposed to the ambient gas and can include at least one electrical or optical property correlative of the target gas composition.

The substrate can be a porous polymer material and the second additive material can include at least one of nanoparticles (NPs), carbon nanotubes (CNTs), sol-gel, a colloidal solution, nanowires, a polymer having a different composition from the porous polymer material, or a combination thereof. For example, the porous polymer material can include a polyvinylidene difluoride (PVDF) support or polyethylene terephthalate (PET). Also, the second additive material can include silica nanoparticles (such as doped within the PVDF or coating the PVDF), multi-walled carbon nano tubes (MWCNTs), polyvinylidene difluoride (PVDF), sol-gel, or a mixture thereof.

Infusion, impregnation, or suspension of the second additive material increases a porosity of the porous polymer material relative to an unimpregnated same porous polymer material. The second additive material can be homogeneously dispersed within the porous polymer material, such that a concentration of the second material within the porous polymer material varies less than 10% throughout a volume of an entire porous polymer material. The second material can also aggregate more when dispersed within a liquid solution in an aqueous phase and aggregates less when the second particle is no longer dispersed within solution and in a non-aqueous phase, such as when suspended in the porous polymer material. Here, impregnation of the second material within the porous polymer material can help reduce aggregation of the second material relative to aggregation of the second material when the second material is in an aqueous phase, such as to help prolong a shelf life of the second material or reduce its deterioration.

In an example, the porous polymer material can be decorated with a non-continuous, sparse uniform coating on a surface thereof, the coating including the second additive material. For example, the second additive material electrosprayed or electrospun onto the porous polymer material. Also, the composite substrate can include a coating at least partially covering at least one of the porous polymer material and the second material from the environment. The porous polymer material can also include at least one of a solid support membrane or a polyvinylidene difluoride (PVDF) membrane. The composite substrate can include a plurality of different functionalized regions, such as an array, an individual functionalized region including at least one first electrical or optical property correlative of a first target gas composition. An individual functionalized region can include, e.g., a colorimetric spot or die. The at least one functionalized region can, in response to an illumination, provide optical response data including spectral response data representing a spectral characteristic of the at least one functionalized region exposed to the ambient gas. For example, the spectral characteristic can include at least one of absorption, reflection, fluorescence, elastic scattering, inelastic (Raman) scattering correlative of a presence or other characteristic of the target gas composition. Also, the at least one functionalized region can include at least one of an oligonucleotide, a metal coordination complex, a porphyrin, a self-assembled monolayer (SAM), a polymer, a pyrrole derivative, a phthalocyanine, or a nanomaterial decoration.

This Summary is intended to provide an overview of the subject matter of the present patent application. It is not intended to provide an exclusive or exhaustive explanation of the invention. The detailed description is included to provide further information.

### BRIEF DESCRIPTION OF THE FIGURES

In the drawings, which are not necessarily drawn to scale, like numerals can describe similar components in different views. Like numerals having different letter suffixes can represent different instances of similar components. The drawings illustrate generally, by way of example, but not by way of limitation, various embodiments discussed in the present document.

FIG. 1A depicts an approach to increasing a coarseness or porosity of a surface of the functionalized region.

FIG. 1B depicts an approach to increasing a coarseness or porosity of a surface of the functionalized region.

FIG. 1C depicts an approach to increasing a coarseness or porosity of a surface of the functionalized region.

FIG. 2A depicts a composite substrate for gas sensing.

FIG. 2B depicts a composite substrate for gas sensing.

FIG. 3A depicts an electrochemical transducer including an array of sensors.

FIG. 3B depicts an electrochemical transducer including a plurality of sensors including a functionalized region.

FIG. 4A depicts an example of an electrochemical transducer including an array of sensors.

FIG. 4B is a detail view of the example of an electrochemical transducer depicted in FIG. 4A.

FIG. 5 is a flowchart that describes a method for optical detection of a target gas composition.

### DETAILED DESCRIPTION

This document describes approaches to detection of a target gas composition within ambient gas in an environment, such as using a gas chemical detector including substrate. A functionalization chemical can be applied to the substrate such as to form a functionalized region. The functionalization chemical can be selected such as to include an optical property indicative of the target gas composition. For example, optical property can be associated with a change of the functionalized region to distinguish the target gas composition from other ambient gases. It can be desirable to utilize a coarseness or porosity of a surface of the functionalized region such as to help increase sensitivity of the gas chemical detector. It can be desirable to have a porous material for a substrate because a porous material can be able to facilitate the detection of small quantities of gases by providing a large surface area for interaction with a chemical sample.

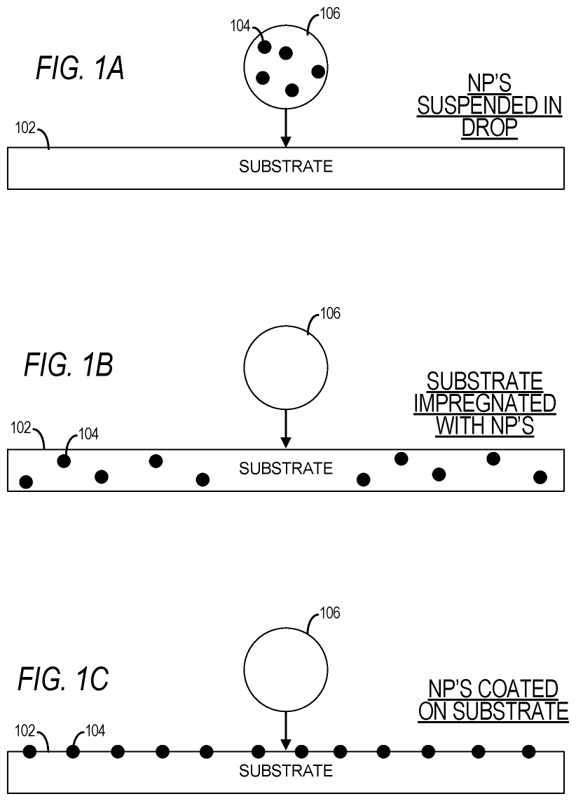

FIG. 1A, FIG. 1B, and FIG. 1C depict several approaches to increasing a coarseness or porosity of a surface of the functionalized region. As shown in FIG. 1A, a functionalization agent 106, such as a fluid drop or spot, can be applied to a substrate 102. The functionalization agent 106 can include at least one chemical property that changes to indicate a presence of a target gas, such as changes in color, absorption, reflectance, fluorescence, phosphorescence, bioluminescence, or Raman emission. It can be ineffective, in certain applications, to merely place the functionalization agent 106 on the substrate 102. To improve responsiveness of the resulting gas chemical sensor, an additive material 104 can be suspended within the functionalization agent 106 before it is applied to the substrate 102. When the functionalization agent 106 is applied to the substrate 102, the additive material 104 can increase a porosity, coarseness, or surface area of the resulting composite material such as to increase a responsiveness or sensitivity of the composite material for gas sensing. A challenge with this approach is that many candidate additive materials 104, e.g., nanoparticles (NPs), carbon nanotubes (CNTs), sol-gel, colloidal solutions, nanowires, lack stability in liquid solution and have a relatively low shelf life. As such, it can be difficult to scale up production of such composite materials for gas sensing while maintaining stability of the additive material 104, especially when several different functionalization agents 106 are each to be applied to the substrate in an array and each including the additive material 104. For example, candidate additive materials such as NPs can involve a shelf life of less than two days in solution with certain functionalization agents 106, sometimes becoming ineffective for increasing porosity, coarseness, or surface area of the composite material after being suspending in the solution for only a few hours. Further, if certain candidate additive materials are left suspended in liquid solution, a sol-gel process can persist until the solution turns to a solid. This can, in certain instances, be catastrophic to preparation of a composite material for gas sensing, such as ruining the functionalization agent 106 before it is able to be applied to the substrate 102. The present inventors have recognized a need for a technique to increase a coarseness or porosity of the substrate 102 without potentially rapidly compromising a solution including a functionalization agent 106.

As shown in FIG. 1B and FIG. 1C, the additive material 104 can be incorporated with the substrate 102 separate from application of the functionalization agent 106. For example, as shown in FIG. 1B, the additive material 104 can be suspended, impregnated, or infused within the substrate 102. For example, an additive material can be added to the substrate 102 separately from the functionalization agent 106 and without impacting a responsiveness of the functionalization agent 106. An additive material 104 can also be added to the substrate after the functionalization agent 106 has been applied to the substrate 102, potentially maintaining a responsiveness or sensitivity of the functionalization agent 106 for a longer duration than otherwise. Suspension, impregnation, or infusion of the additive material 104 within the substrate 102 can be performed at a temperature and pressure that is effective to cause the additive material 104 to disperse and create a uniform distribution of the additive material 104 throughout the substrate 102. As shown in FIG. 1C, the additive material 104 can also be coated onto a surface of the substrate 102. For example, the additive material 104 can be sprayed, spin-coated, or incorporated via a dipping process. In an example, such as to help evenly coat the additive material 104, the surface of the substrate 102 can be first wet with a liquid, e.g., water, and then subsequently coated. Also, the surface of the substrate can be pre-treated, e.g., cleaned or polished, before the additive material 104 is coated thereon.

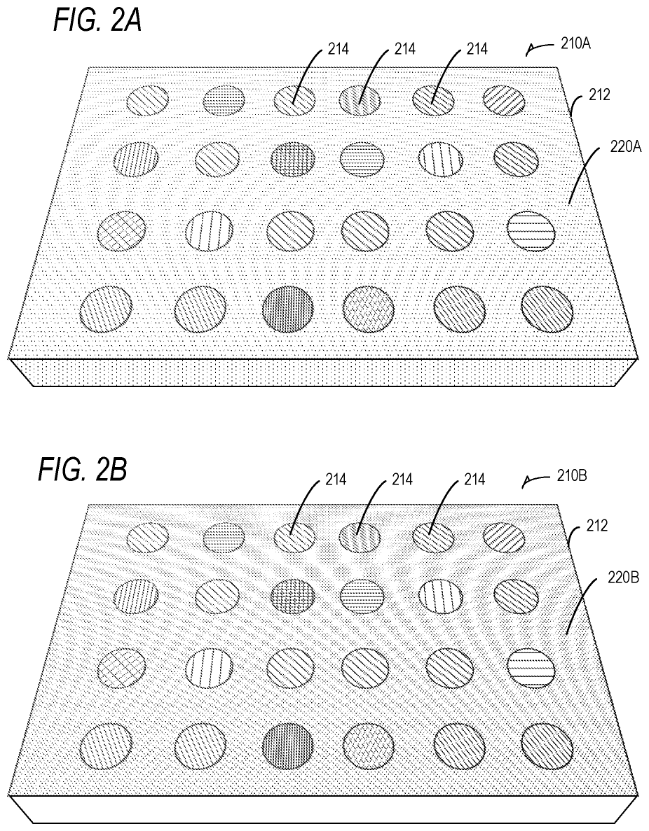

FIG. 2A and FIG. 2B each depict an example of a composite substrate for gas sensing. As depicted in FIG. 2A, a composite substrate 210A can include a porous polymer material 212 incorporating a second additive material 220A. The composite substrate 210 can also include at least one functionalized region 214, included such as to be exposed to the ambient gas. The second additive material 220A can include at least one chemical property to establish or adjust a hydrophobicity of the porous polymer material 212. For example, infusion, impregnation, or suspension of the second additive material 220A can increase a porosity of the porous polymer material 212 relative to an unimpregnated same porous polymer material 212. The second additive material 220A can be homogeneously dispersed within the porous polymer material 212, such that a concentration of the second additive material 220A within the porous polymer material 212 varies less than 10% throughout a volume of an entire porous polymer material. The second additive material can include a 1 dimensional or 2 dimensional nanomaterial. For example, the second additive material can include nanoparticles (NPs), carbon nanotubes (CNTs), sol-gel, a colloidal solution, nanowires, graphene, millennium disulphide, or a polymer different from the porous polymer material. The second additive material 220A can include silica nanoparticles doped within a polyvinylidene difluoride PVDF porous polymer material 212 or silica nanoparticles coating the PVDF. The second additive material 220A can include a mixture of PVDF and sol-gel. The second additive material 220A can include multi-walled carbon nano tubes 224 (MWCNTs) impregnated within the PVDF. In an example, the second additive material 220 can tend to aggregate more in an aqueous phase, such as when suspended in a liquid solution, than the second additive material tends to aggregate embedded in or on the porous polymer material 212.

The porous polymer material 212 can include a solid support membrane or a polyvinylidene difluoride (PVDF) membrane. For example, the porous polymer material 212 can include a first PVDF layer and a second PVDF layer. The first PVDF layer 230 can include a greater density than the second PVDF layer and the second additive material 220A can be incorporated within the second PVDF layer 216. In an example, the porous polymer material 212 can include polyethylene terephthalate (PET).

In an example, the at least one functionalized region 214 can include an array of different functionalized regions. In an example, the at least one functionalized region 214 can include at least one colorimetric spot. In an example, the at least one functionalized region 214 can be arranged to, in response to an illumination, provide optical response data. The optical response data may include at least one of absorption, reflection, fluorescence, elastic scattering, inelastic (Raman) scattering correlative of a presence or other characteristic of the target gas composition. In an example, the at least one functionalized region 214 may include at least one of an oligonucleotide, a metal coordination complex, a porphyrin, a self-assembled monolayer (SAM), a polymer, a pyrrole derivative, a phthalocyanine, or a nanomaterial decoration.

FIG. 2B depicts a similar a composite substrate 210B to composite substrate 210A described above with respect to FIG. 2A. As such, like elements of composite substrate 210B can be similar to those described above. Here, the composite substrate 210B can include a porous polymer material 212 decorated with a non-continuous, sparse uniform coating, including the second additive material 220B, on a surface thereof. For example, the second additive material 220B can be at least one of electrosprayed material or an electrospun material onto the porous polymer material 212. Here, a concentration of the second additive material 220B within a volume of the porous polymer material 212 can be non-uniform, including a concentration gradient such that the second additive material 220B is more present on a surface the porous polymer material 212 relative to a center of the porous polymer material 212. While here the second additive material 220B is not uniformly dispersed within a volume of the porous polymer material, it can be uniformly coated across a surface of the porous polymer material. For example, the second additive material 220B can be not localized at any particular functionalized region 214. The composite substrate can also include or use a protective coating at least partially covering at least one of the porous polymer material 212 or the second additive material 220A from the environment. The protective coating can include the second additive material 220A, or can be deposited over the top of second additive material 220 which has been previously decorated atop the porous polymer material 212.

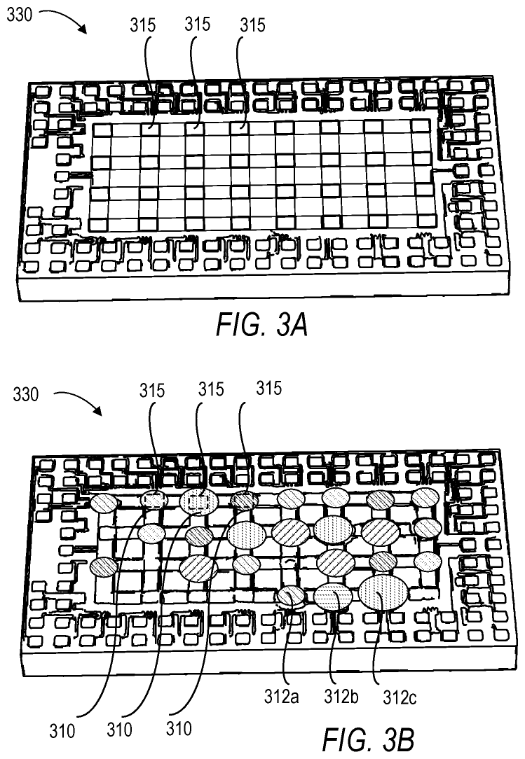

FIG. 3A depicts an electrochemical transducer including a composite substrate material. Composite substrates described herein can be used within electrochemical sensors of a transducer, such as for sensing of a target gas in an ambient environment. An electrochemical transducer 330 can include at least one sensor 315, such as an array of sensors 315. Here, an individual sensor can include or use a composite substrate as described above with respect to FIG. 1A, FIG. 1B, FIG. 2A, and FIG. 2B. Also, an individual sensor 315 can include an electrochemical sensing element such as a Field Effect Transistor (FET), a p-n junction diode, an electrochemical cell, a conductive polymer or electrode, or other semiconductor or other device that, when exposed to a particular target gas composition, changes a signal (e.g., current, voltage, or impedance) in accordance with a presence or concentration of the target gas composition.

FIG. 3B depicts the electrochemical transducer of FIG. 3A including several functionalization agents applied to a composite substrate of an individual sensor. In an example, an individual sensor 315 can include or use at least one functionalized region 310. The functionalized region 310 can include a covering or coating including at least one functionalization agent 312 over the electrochemical sensing element of an individual sensor 315. Also, the at least one functionalization agent 312 can be otherwise present at or formed with the electrochemical sensing element of an individual sensor 315. As depicted in FIG. 3B, the electrochemical transducer 330 can include a plurality of sensors 315 respectively including different functionalization agents such as functionalization agents 312a, 312b, and 312c. In an example, the different functionalization agents can include an oligonucleotide, a metal coordination complex, a porphyrin, a self-assembled monolayer (SAM), a polymer, a pyrrole derivative, a phthalocyanine, or a nanomaterial decoration.

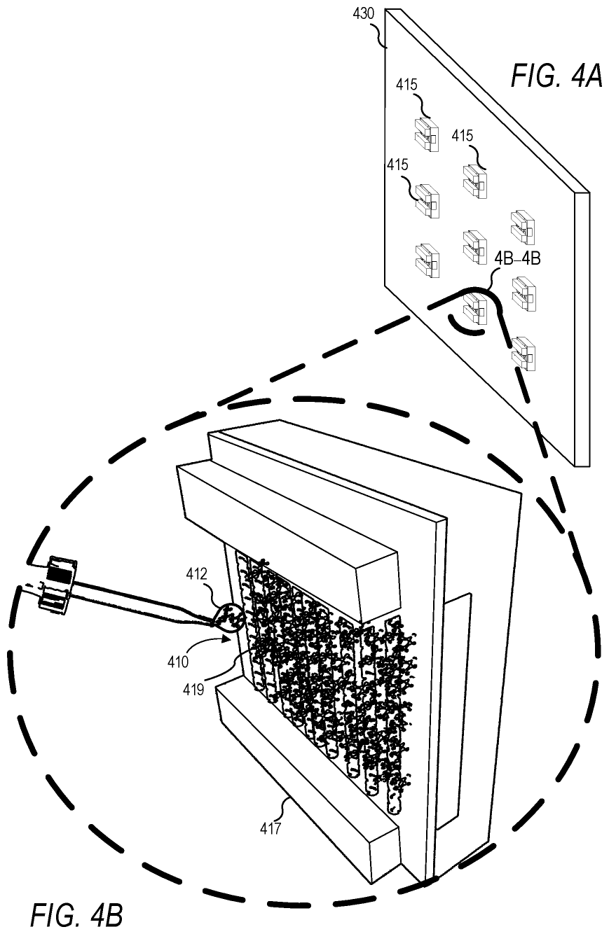

FIG. 4A and FIG. 4B depict another example of an electrochemical transducer 430 including an array of sensors. The electrochemical transducer 430 depicted in FIG. 4A can be similar to that described above with respect to electrochemical transducer 330 depicted in FIG. 3A. As such, like elements can be to those described above. An array of sensors 415 of an electrochemical transducer 430 can each include respective CNFETs 417. In the detailed view as shown in FIG. 4B, at least one functionalization agent 412 can be applied to an individual sensor 415 such as at a CNFET 417 of the sensor 415. Application of the functionalization agent 412 can include, e.g., physical evaporation, chemical vapor deposition, photolithography, spin-coating, or dip-coating of the functionalization agent 412 at a functionalized region 410 the sensor 415. For example, a monolayer, bilayer, or multi-layer of a functionalization agent 412 can be applied to the CNTs 419 of an individual CNFET 417.

As described above, certain ones of the functionalization agents 412 can include optically detectable characteristics to help detect the target gas composition. For example, the functionalization agent 412 can be optically detectable, or luminescent, such that it emits light upon exposure to the particular target gas composition. For example, the functionalization agent 412 can include colorimetric indicator molecules that can change color or emit fluorescent light when exposed to a particular target gas composition.

The array of sensors 415 can be functionalized with a plurality of different functionalization agents 412. For example, the array of sensors 415 can include at least one functionalization agent 412 including an optically detectable characteristic to help detect the target gas composition and at least one electrically detectable characteristic to help detect the same target gas composition. In another example, the array of sensors 415 can be functionalized with a plurality of functionalization agents 412 including optically or electrically detectable characteristics to help detect at least two different target gas compositions. Here, the array of sensors 415 can concurrently and individually screen for a presence or other characteristic of multiple target gases or multiple target gas compositions.

Functionalization agents 412 for application at an individual functionalized region 410 of the electrochemical transducer 430 can include, e.g., oligonucleotides, metal coordination complexes, porphyrins, self-assembled monolayers (SAMs), polymers, pyrrole derivatives, phthalocyanines, nanomaterial decorations, biotin-avidin linkages, peptides, antibodies, enzymes, or one or more combinations thereof. Also, certain functionalization agents 412 can be included in the electrochemical sensor, such as at the functionalized region 410, for optical signal transducing and digitization before and after exposure to a target gas. Such functionalization agents can include, e.g., antibodies, polymer probes, aptamers, micro-particles with molecular targets, metal particles, fluorescent materials, silica microspheres, silica/polymer hybrid microspheres, nanocomposites with magnetic, noble, or semiconductor nanoparticles, or one or more combinations thereof.

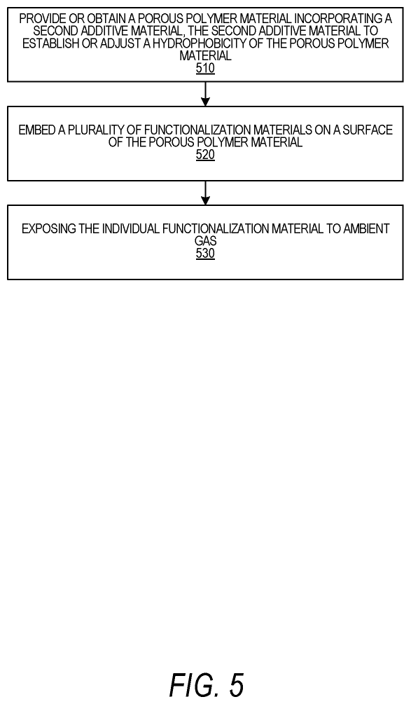

FIG. 5 is a flowchart that describes a method for optical detection of a target gas composition. In an example, at 510, the method can include providing or obtaining a porous polymer material incorporating a second additive material, the second additive material including at least one chemical property to establish or adjust a hydrophobicity of the porous polymer material. For example, the porous polymer material can be impregnated with the second material including at least one of nanoparticles (NPs), carbon nanotubes (CNTs), sol-gel, a colloidal solution, nanowires, a polymer having a different composition from the porous polymer material, or a combination thereof. In an example, the method can include increasing a porosity of the porous polymer material relative to an unimpregnated same porous polymer material. In an example, the method can include homogeneously dispersing the second material within the porous polymer material, such that a concentration of the second material within the porous polymer material varies less than ten percent throughout a volume of an entire porous polymer material. In an example the method can include decorating the porous polymer material with a non-continuous, sparse uniform coating on a surface thereof, the coating. Also, the method can include electrospraying material or electrospinning the second material onto the porous polymer material.

At 520, the method can include embedding a plurality of functionalization agents on a surface of the porous polymer material, an individual functionalization agent of the plurality of functionalization agents including at least one electrical or optical property correlative of the target gas composition.

At 530, the method can include exposing the individual functionalization agent to ambient gas.

The above Detailed Description can include references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “including” and “in which” are used as the plain-English equivalents of the respective terms “comprising” and “wherein.” Also, in the following aspects, the terms “including” and “comprising” are open-ended, that is, a system, device, article, composition, formulation, or process that can include elements in addition to those listed after such a term in a aspect are still deemed to fall within the scope of that aspect.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” can include “A but not B,” “B but not A,” and “A and B,” unless otherwise indicated. In this document, the terms “including” and “in which” are used as the plain-English equivalents of the respective terms “comprising” and “wherein.” Also, in the following aspects, the terms “including” and “comprising” are open-ended, that is, a system, device, article, composition, formulation, or process that can include elements in addition to those listed after such a term in a aspect are still deemed to fall within the scope of that aspect. Moreover, in the following aspects, the terms “first,” “second,” and “third,” etc. are used merely as labels, and are not intended to impose numerical requirements on their objects.

The above description is intended to be illustrative, and not restrictive. For example, the above-described examples (or one or more aspects thereof) can be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the aspects. Also, in the above Detailed Description, various features can be grouped together to streamline the disclosure. This should not be interpreted as intending that an unclaimed disclosed feature is essential to any aspect. Rather, inventive subject matter can lie in less than all features of a particular disclosed embodiment. Thus, the following aspects are hereby incorporated into the Detailed Description as examples or embodiments, with each aspect standing on its own as a separate embodiment, and it is contemplated that such embodiments can be combined with each other in various combinations or permutations. The scope of the invention should be determined with reference to the appended aspects, along with the full scope of equivalents to which such aspects are entitled.

What is claimed is:

1. A composite substrate for optical detection of a target gas composition within ambient gas in an environment, the composite substrate comprising:
a porous polymer material incorporating a second material, the second material including at least one chemical property to establish or adjust a hydrophobicity of the porous polymer material; and
at least one functionalized region, configured to be exposed to the ambient gas, at least partially embedded in the porous polymer material, the functionalized region including at least one electrical or optical property correlative of the target gas composition.

2. The composite substrate of claim 1, wherein the porous polymer material is impregnated with the second material including at least one of nanoparticles (NPs), carbon nanotubes (CNTs), graphene, sol-gel, a colloidal solution, nanowires, a polymer having a different composition from the porous polymer material, or a combination thereof.

3. The composite substrate of claim 2, wherein impregnation of the second material increases a porosity of the porous polymer material relative to an unimpregnated same porous polymer material.

4. The composite substrate of claim 2, wherein the second material is homogeneously dispersed within the porous polymer material, such that a concentration of the second material within the porous polymer material varying less than ten percent throughout a volume of an entire porous polymer material.

5. The composite substrate of claim 2, wherein the second particle aggregates more when dispersed within a liquid solution in an aqueous phase and aggregates less when the second particle is no longer dispersed within solution and in a non-aqueous phase.

6. The composite substrate of claim 1, wherein the porous polymer material is decorated with a non-continuous, sparse uniform coating on a surface thereof, the coating including the second material.

7. The composite substrate of claim 6, wherein the second material is at least one of an electrosprayed material or an electrospun material onto the porous polymer material.

8. The composite substrate of claim 6, wherein the second material includes at least one of nanoparticles (NPs), carbon nanotubes (CNTs), graphene, sol-gel, a colloidal solution, nanowires, a polymer having a different composition from the porous polymer material, or a combination thereof.

9. The composite substrate of claim 8, comprising a coating at least partially covering at least one of the porous polymer material and the second material from the environment.

10. The composite substrate of claim 9, wherein the coating includes the second material.

11. The composite substrate of claim 1, wherein the porous polymer material includes at least one of a solid support membrane or includes a membrane comprised of at least 90% a polyvinylidene difluoride (PVDF) by weight.

12. The composite substrate of claim 1, wherein:
the at least one functionalized region includes a plurality of different functionalized regions including:
a first functionalized region including at least one first electrical or optical property correlative of a first target gas composition; and
a second functionalized region including at least one second electrical or optical property correlative of a second target gas composition;
wherein the at least one second electrical or optical property is different from the first at least one first electrical or optical property.

13. The composite substrate of claim 1, wherein the at least one functionalized region includes an array of different functionalized regions.

14. The composite substrate of claim 1, wherein the at least one functionalized region includes at least one colorimetric spot.

15. The composite substrate of claim 1, wherein the porous polymer material includes a polyvinylidene difluoride (PVDF) support and the second material includes silica nanoparticles doped within the PVDF.

16. The composite substrate of claim 1, wherein the porous polymer material includes polyvinylidene difluoride (PVDF) and the second material includes multi-walled carbon nano tubes (MWCNTs) impregnated within the PVDF.

17. The composite substrate of claim 1, wherein the porous polymer material includes polyethylene terephthalate (PET), and the second material includes a mixture of polyvinylidene difluoride (PVDF) and sol-gel.

18. The composite substrate of claim 1, wherein the porous polymer material includes a polyvinylidene difluoride (PVDF) support and the second material includes silica nanoparticles coating the PVDF.

19. The composite substrate of claim 1, wherein the porous polymer material includes a first polyvinylidene difluoride (PVDF) layer and a second PVDF layer, wherein the first PVDF layer has a greater density than the second PVDF layer and the second material is incorporated within the second PVDF layer.

20. The composite substrate of claim 1, wherein the at least one functionalized region is arranged to, in response to an illumination, provide optical response data including spectral response data representing a spectral characteristic of the at least one functionalized region exposed to the ambient gas, the spectral characteristic including at least one of absorption, reflection, fluorescence, elastic scattering, inelastic (Raman) scattering correlative of a presence or other characteristic of the target gas composition.

21. The composite substrate of claim 1, wherein the at least one functionalized region includes at least one of an oligonucleotide, a metal coordination complex, a porphyrin, a self-assembled monolayer (SAM), a polymer, a pyrrole derivative, a phthalocyanine, or a nanomaterial decoration.

22. A method for optical detection of a target gas composition within ambient gas in an environment, the method comprising:
providing or obtaining a porous polymer material incorporating a second material, the second material including at least one chemical property to establish or adjust a hydrophobicity of the porous polymer material;
embedding a plurality of functionalization agents on a surface of the porous polymer material, an individual functionalization agent of the plurality of functionalization agents including at least one electrical or optical property correlative of the target gas composition
exposing the individual functionalization agent to ambient gas.

23. The method of claim 22, wherein the porous polymer material is impregnated with the second material including at least one of nanoparticles (NPs), carbon nanotubes (CNTs), sol-gel, a colloidal solution, nanowires, a polymer having a different composition from the porous polymer material, or a combination thereof.

24. The method of claim 23, comprising increasing a porosity of the porous polymer material relative to an unimpregnated same porous polymer material.

25. The method of claim 23, comprising homogeneously dispersing the second material within the porous polymer material, such that a concentration of the second material within the porous polymer material varies less than ten percent throughout a volume of an entire porous polymer material.

26. The method of claim 23, comprising:
reducing aggregation, via impregnation of the second material within the porous polymer material, of the second material relative to aggregation of the second material when the second material is in an aqueous phase;
wherein the second particle aggregates more when dispersed within a liquid solution in an aqueous phase and aggregates less when the second particle is no longer dispersed within solution and in a non-aqueous phase.

27. The method of claim 22, comprising decorating the porous polymer material with a non-continuous, sparse uniform coating on a surface thereof, the coating including the second material.

28. The method of claim 27, comprising electrospraying material or electrospinning the second material onto the porous polymer material.

29. The method claim 27, wherein the second material includes at least one of nanoparticles (NPs), carbon nanotubes (CNTs), sol-gel, a colloidal solution, nanowires, a polymer having a different composition from the porous polymer material, or a combination thereof.
